SEQUENCE LISTING (5 sequences)

SEQ ID NO: 1 - DNA, 50 bp, Artificial
  Synthetic oligonucleotide
tgcaggtcgactgtctcttatacacatcttgagtgagtgagcatgcatgt

SEQ ID NO: 2 - DNA, 60 bp, Artificial
  Synthetic oligonucleotide
ggccacgacacgctcccgcgctgtctcttatacacatcttgagtgagtgagcatgcatgt

SEQ ID NO: 3 - DNA, 19 bp, Artificial
  Synthetic oligonucleotide
ctgtctcttatacacatct

SEQ ID NO: 4 - DNA, 40 bp, Artificial
  Synthetic oligonucleotide
ctgtctcttatacacatcttgagtgagtgagcatgcatgt

SEQ ID NO: 5 - DNA, 60 bp, Artificial; identical to SEQ ID NO: 2
  Synthetic oligonucleotide

(19) United States
(12) Patent Application Publication
(10) Pub. No.: US 2006/0035245 A1
(43) Pub. Date: Feb. 16, 2006

(54) Modulators of enzymatic nucleic acid elements mobilization

(76) Inventors: Brandon L. Ason, Utrecht (NL); William S. Reznikoff, Madison, WI (US); Anna M. Skalka, Princeton, NJ (US); Daniel Knauss, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/110,465

(22) Filed: Apr. 20, 2005

Related U.S. Application Data
(60) Provisional application No. 60/563,602, filed on Apr. 20, 2004.

(51) Int. Cl. C12Q 1/68

(52) U.S. Cl. 435/6

(57) ABSTRACT

The present invention discloses a nucleic acid cleavage assay for members of the transposase/integrase superfamily. A method of using the assay to screen for modulators of the nucleic acid cleavage activity is also disclosed. The present invention further provides a method for screening for modulators of binding of a transposase/integrase to its corresponding recognition sequence. In addition, the present invention provides a method of identifying a modulator for a particular transposase/integrase such as HIV integrase based on modulators of other members of the transposase/integrase superfamily. Also disclosed are Tn5 transposase inhibitors and HIV integration inhibitors.

### CROSS-REFERENCE TO RELATED APPLICATIONS

This application claims the benefit of U.S. provisional patent application Ser. No. 60/563,602, filed on Apr. 20, 2004, incorporated by reference in its entirety.

### STATEMENT REGARDING FEDERALLY SPONSORED RESEARCH OR DEVELOPMENT

The present invention was made with United States government support awarded by the following agency: National Institutes of Health, Grant No. GM050692. The United States government has certain rights in this invention.

### BACKGROUND OF THE INVENTION

Members of the transposase/integrase protein superfamily are involved in sequence specific binding to the end of a related transposon/retroviral DNA followed by DNA nicking or cleavage and mobilization. For example, a transposase encoded by Tn5 transposon in the IS4 family of prokaryotic transposable elements is responsible for Tn5 transposition into a nucleic acid target. Likewise, another member of the superfamily, human immunodeficiency virus (HIV) integrase, is responsible for integration of HIV into a target genome. The transposase and integrase proteins share little primary sequence identity but extensive functional and three dimensional structural identity, especially in the catalytic core having an alpha-beta-alpha fold with three conserved acidic amino acid residues responsible for divalent metal coordination required for catalysis. In a native spatial conformation, the three catalytic residues (known as the DDE motif) are located very close to one another. In Tn5 transposase, the three acidic residues are D97, D188 and E326. For HIV integrase, the residues are D64, D116 and E152.

DNA transposition/integration catalyzed by an enzyme of the transposase/integrase superfamily involves, among other steps, the formation under suitable conditions of a bound DNA-enzyme complex and the catalytic cleavage of DNA by the enzyme. For example, Tn5 transposase-mediated transposition steps include (1) binding of Tn5 transposase to a transposase recognition sequence of a Tn5 transposon, (2) formation of a synaptic complex, (3) DNA strand cleavage, and (4) insertion of the transposon DNA into the target by strand transfer. Catalytic cleavage cannot occur if the enzyme cannot bind to the recognition sequence or if the catalytic activity of the enzyme is inhibited.

In view of the close structural and functional similarities among proteins of the transposase/integrase superfamily, it is likely that compounds that interact with one protein would also interact with other proteins in the family. It would be desirable to identify modulators of Tn5 transposase that inhibit (or enhance) integration activity of HIV or other integrase proteins. The art is in need of modulators of members of this superfamily for research purposes associated with understanding the activities of transposase and integrase, and has particular need for inhibitors as pharmacological therapeutic agents for reducing or preventing retroviral integration catalyzed by the proteins, particularly integration into the human genome by HIV-1.

U.S. Pat. No. 5,786,139, incorporated herein by reference as if set forth in its entirety, disclosed a method of detecting enzymatic nucleic acid cleavage activity using fluorescence polarization (FP). The method relies on the difference in FP between the fluorescently labeled parent nucleic acid (with no nuclease attached) and its degradation products. U.S. Pat. No. 5,786,139 did not contemplate either modulating the enzyme activity to alter cleavage or using FP to identify a change in cleavage activity.

U.S. Pat. No. 5,763,181 disclosed using fluorometric assays for detecting nucleic acid cleavage. In particular, the patent acknowledges the ability to use fluorometric methods to measure the effectiveness of specific inhibitors on HIV integrase or other retroviral integrase proteins. However, the inventors of the '181 patent do not employ FP. Rather, the inventors monitor relative fluorescence intensity.

U.S. Pat. No. 6,589,744 uses FP to detect modulators of ligase and of helicase enzyme activity, but is not concerned with modulators of binding or cleavage activities.

### BRIEF SUMMARY OF THE INVENTION

The present invention discloses a nucleic acid (e.g., DNA) cleavage assay for members of the transposase/integrase superfamily. A method of using the assay to screen for modulators of the nucleic acid cleavage activity is also disclosed. The present invention further provides a method for screening for modulators of binding of a transposase/integrase to its corresponding recognition sequence. In addition, the present invention provides a method of identifying a modulator for a particular transposase/integrase such as HIV integrase based on modulators of other members of the transposase/integrase superfamily. Also disclosed are Tn5 transposase inhibitors and HIV integration inhibitors.

### BRIEF DESCRIPTION OF THE SEVERAL VIEWS OF THE DRAWINGS

FIG. 1 is a schematic representation of the fluorescently labeled non-transferred DNA strand and the FP assay. A) The dsDNA fragments used for the FP assay were 50 nt in length and contain a 10 nt donor DNA region followed by a 40 nt transposon DNA region. The 10 nt donor DNA region is released following the production of a double strand break accompanying strand cleavage. B) This FP assay measures the change in tumbling rate of a population of fluorescently labeled DNA molecules. A fluorescently labeled DNA fragment either free or complexed with transposase has a particular tumbling rate in solution. This tumbling rate is measured by the change in polarized fluorescence intensity between the time of fluorophore excitation and emission. In this application, the apparent tumbling rate increases as the number of cleaved donor DNA fragments increases. Thus, any transposase effector that inhibits strand cleavage without affecting transposase-DNA binding interactions would in effect increase the observed FP value compared to the value for either the cleaved donor DNA fragments or the free DNA substrate. Tnp=transposase.

FIG. 2 shows that a comparison of FP and gel-shift assays indicates that FP is a good measurement of the degree of Tn5 transposase induced strand cleavage. In order to access the relationship between FP and strand cleavage, we compared the degree of strand cleavage that occurred as a function of various EDTA concentrations under the same conditions. Increasing concentrations of EDTA (0, 0.01, 0.05, 0.1, 0.5, 1, 2, 3, 4, 5, 10, 20, 50 mM) were used as a mock inhibitor during assay development. A) Strand cleavage reactions were incubated at 37° C. for 1.5 h and subsequently loaded onto a 9% native gel for gel-shift assays. The DNA was detected using 5′ fluorescein labeled oligonucleotides. Bd sub indicates the transposase-DNA substrate complex, bd prod indicates the transposase-DNA product complex, DNA sub is the substrate, and cleaved prod is the donor DNA cleavage product. The graph represents the percentage of DNA cleavage products compared to the amount of DNA bound by the transposase per lane from data collect from multiple gel shift assays. B) Strand cleavage reactions were incubated at 37° C. for 1.5 h prior to the FP measurement. The oligonucleotides were labeled with rhodamine green on the 5′ end of the non-transferred strand for these experiments. The graph represents the change in FP signal with increasing concentrations of EDTA. These data indicate that the polarization and strand cleavage data are in good agreement, since the concentration of EDTA that inhibits cleavage is along the same order of magnitude that the polarization begins to increase.

FIG. 3 shows that strand cleavage inhibition is distinguishable from complex assembly inhibition. Transposase complexed with fluorescently labeled DNA exhibits a significantly higher FP value than either the free DNA substrate (mock complex assembly inhibition) or the cleaved donor DNA product (no inhibition). Reactions were performed as originally described in FIG. 2 and the materials and methods in example 1 below. Transposase storage buffer was added to the DNA substrate in place of transposase for mock complex assembly inhibition. For catalytic inhibition, 10 mM EDTA was added to the cleavage reaction. Reactions were performed in triplicate. Tnp=transposase.

FIG. 4 is a comparison of FP and gel shift synapsis assays. Fluorescence polarization (FP) is a good measurement of the amount of synaptic complexes being formed. Increasing concentrations of unlabeled DNA (0.001, 0.005, 0.01, 0.05, 0.1, 0.5, 1, 2, 5, 8, 10, 20, 50 μM) was used as a competitive inhibitor for synaptic complex formation using 160 nM fluorescently labeled DNA and 800 nM transposase. FP data (bottom) was compared to data obtained using gel shift assays (top). Bands labeled hetero DNA-transposase represent two dsDNA molecules, one fluorescently labeled and one unlabeled. The lane labeled ‘no comp.’ does not contain any unlabeled competitor DNA. IC₅₀ values were obtained from fitting these data to an exponential decay and determining the concentration of unlabeled DNA that reduced the amount of fluorescently labeled DNA by one half. IC₅₀ values for FP and gel shift assays are 2.6±0.2 μM and 5.1±0.4 μM respectively, suggesting that FP is a good measurement of the degree of synaptic complex formation. These data were obtained from multiple experiments. Each data point represents at least two, typically three, independent experiments. Error is represented as the standard deviation (SD) from the fit of the data to the equation used to calculate the IC₅₀. Tnp=transposase.

FIG. 5 shows gel-shift assay results that verify Tn5 transposase inhibition. In this example, an aromatic thiourea is shown to be a modest inhibitor of transposase-DNA complex assembly with an IC₅₀ of 24 μM. These values are reported in FIG. 9. The gel shift illustrates compound inhibition of transposase-DNA complexes in a typical assay and is representative of the data collected for the graph, which was used to calculate an IC₅₀ for the inhibitor. The gel shift observed at low and high inhibitor concentrations are identical to the ones observed for the DMSO only and no transposase control reactions respectively. The data in this graph were obtained from multiple experiments. Each data point represents at least two, typically three, independent experiments. Error is represented as the standard deviation (SD) from the fit of the data to the equation used to calculate the IC₅₀. Tnp=transposase.

FIG. 6 shows twenty compounds that inhibit Tn5 transposase-DNA assembly. Several substructures consisting of coumarin, benzoic acid and cinnamoyl derivatives were identified within this group. These compounds range in IC₅₀ values from 3.5 to 46 μM. IC₅₀ values were determined from gel shift assays, similar to FIG. 5. These data were obtained from multiple experiments. Each value represents at least two, typically three, independent experiments. Error is represented as the standard deviation (SD) from the fit of the data to the equation used to calculate the IC₅₀.

FIG. 7 shows that a large coumarin dimer inhibits integrase activity with an IC₅₀ of 15 μM. IC₅₀s were determined by measuring the degree of 3′ strand processing that occurs with increasing concentrations of inhibitor (0, 0.05, 0.5, 5, 25, 50, 75, 100, 150, 250, 375, or 500 μM) added to the integrase reactions, as described in the methods section. The degree of 3′ strand processing is measured by the percentage of the signal obtained for the 3′ strand processing product relative to the total signal per lane. These values are reported in FIG. 8. IN=integrase.

FIG. 8 shows that 6 compounds consisting of a biothionol, coumarin and cinnamoyl derivatives inhibit HIV-1 integrase activity. These compounds range in IC₅₀ values from 9 to 32 μM. These values are calculated from data measuring the percentage of the 3′ strand processing product formed, as described in FIG. 6. Assay products are observed using PAGE, as described in the methods section. Each value represents at least two, typically three, independent experiments. Error is represented as the standard deviation (SD) from the fit of the data to the equation used to calculate the IC₅₀.

FIG. 9 shows the relationship between structure and activity for several related compounds reveals two regions that impact cytotoxicity and HIV integration in cells. Increasing concentrations (0.01, 0. 1, 1, 10, 20, 30, 50, 75, 100 μM) of each compound were used to determine their ability to block HIV-1 integration in vivo and determine their inherent cytotoxicity, as described in the methods section of example 2 below. Reactions were performed in triplicate. Each compound is further analyzed for inhibition of integrase activity in vitro, as described in FIG. 6. Each value represents three independent experiments. Error is represented as the standard deviation (SD) from the fit of the data to the equation used to calculate the IC₅₀. IN=integrase.

FIG. 10 shows that inhibitory compounds block HIV-1 transduction in cells at a point in the viral lifecycle consistent with inhibition of integration. The inhibitory compounds were simultaneously assayed for their effects on reverse transcription, transduction, gross cell viability, and expression of a stably-integrated luciferase-encoding provirus. A QPCR assay was used to monitor effects of the compounds on reverse transcription in acutely infected cells. The compounds were also measured in parallel for their effects on general cell viability and for their effects on expression of luciferase encoded from proviral DNA in chronically infected cells, as described in materials and methods. The results demonstrated that compounds 10, 10-B, and 10-F have no significant inhibitory effect on either reverse transcription (or any earlier step in the viral lifecycle), cell viability, or luciferase expression. Reactions were performed in triplicate using 75 μM of each inhibitor.

### DETAILED DESCRIPTION OF THE INVENTION

The present invention uses a FP (a term used interchangeably herein with “fluorescence anisotropy”) method to identify compounds, such as small organic compounds, that can modulate (inhibit or enhance) binding of a member of the transposase/integrase superfamily to a target nucleic acid, cleavage of the nucleic acid, or both. Examples of members of the transposase/integrase superfamily include but are not limited to Tn5 transposase, Tn10 transposase, Mu transposase, and retroviral integrases such as HIV integrase and avian sarcoma virus integrase. Preferably and most efficiently, the compounds are identified from a large library of compounds, such as any of several commercially available libraries.

The method of the present invention is based on the principle that molecules of different sizes rotate at different speeds and thus have different levels of FP if labeled with a fluorescent tag. Therefore, the binding of a transposase/integrase to a target DNA molecule can be detected by fluorescently labeling the DNA molecule and observing a change in FP from that of the smaller target DNA molecule to that of the larger transposase/integrase-DNA complex. Likewise, the cleavage of the DNA molecule can be detected by a change in FP from that of the larger transposase/integrase-DNA complex to that of a smaller DNA cleavage product that carries the fluorescent label. It will become apparent in the examples below that the method of the present invention involves no washing step and requires minimal sample volume. Therefore, they can be easily adapted for high throughput applications.

In view of the recognized structural and functional similarities among members of the transposase/integrase superfamily (Bhasin, A. et al., J Biol Chem 274: 37021-37029, 1999; Kennedy, A. K. et al., Cell 95: 125-134, 1998; Mizuuchi, K. and Adzuma, K., Cell 66: 129-140, 1991; Engelman, A. et al., Cell 67: 1211-1221, 1991; Davies, D.R. et al., J Biol Chem 274: 11904-11913, 1999; Dyda, F. et al., Science 266: 1981-1986, 1994; Bujacz, G. et al., J Mol Biol 253: 333-346, 1995; Rice, P. and Mizuuchi, K., Cell 82: 209-220, 1995; Doak, T. G. et al., Proc Natl Acad Sci USA 91: 942-946, 1994; and Davies, D. R. et al., Science 289: 77-85, 2000, each of which is herein incorporated by reference in its entirety), the identified modulators of one member are considered candidate modulators of other members and can thus be tested for an ability to modulate the activity of other members. The identified inhibitors having anti-integrative activities can be formulated in a pharmaceutical composition comprising a carrier for therapeutic administration to reduce or prevent integration (transposition) into the human genome of HIV and other transposable nucleic acid. In this regard, it is of particular interest to use the methods of the invention to identify compounds having an integration-inhibiting activity against HIV integrase. The identified inhibitors can also be used to trap intermediates of transposase/integrase reactions. The structures of these intermediates can be studied, for example, by co-crystallization to facilitate rational drug design.

Additionally, modulators of a member of the transposase/integrase superfamily may also affect other proteins with nucleic acid recombination activities, such as the RAG (recombination activating genes) recombinase complex involved in V[D]J recombination, which has a three-amino acid functional cleavage site similar to the members of the transposase/integrase superfamily (Landree, M. A. et al., Genes and Development 13:3059-3069, 1999, incorporated herein by reference in its entirety).

In a preferred embodiment, the methods of the present invention are practiced with a transposase (e.g., Tn5 transposase) and the transposase modulators identified are further tested for their modulating activities on other transposases and integrases (e.g., HIV integrase) as well as other proteins with nucleic acid recombination activities.

Transposase/Integrase Cleavage Assay and Screening for Cleavage Modulators

In one aspect, the present invention relates to a method for observing the nucleic acid (e.g., DNA) cleavage activity of a transposase or integrase. The method includes the steps of:

a) incubating a transposase/integrase protein and a nucleic acid molecule that comprises a recognition sequence of the transposase/integrase under conditions that allow the transposase/integrase to bind, specifically and non-covalently, to the recognition sequence and cleave the nucleic acid molecule wherein the nucleic acid molecule is labeled with a fluorescent label at a position such that upon the cleavage a fragment not bound by the transposase/integrase and carries the fluorescent label is released;

b) measuring the FP of the label either during or following step a); and

c) comparing the FP measurement of step b) to that of at least one control to determine the cleavage of the nucleic acid molecule.

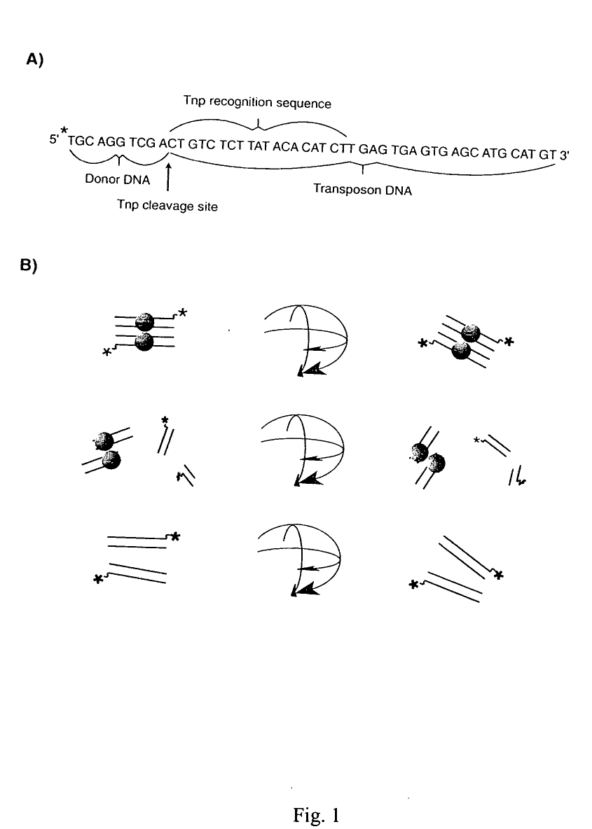

It is noted that the cleavage assay of the present invention employs a model system of two molecules of transposase/integrase enzyme and two nucleic acid fragments, with each fragment comprising one transposase/integrase recognition sequence (FIG. 1). In contrast, an authentic transposase/integrase nucleic acid complex comprises two transposase/integrase protein molecules bound to one nucleic acid fragment that comprises two recognition sequences. Use of such a model system advantageously facilitates identification of modulators of transposase and integrase cleavage activity. For step a), the transposase/integrase protein and the nucleic acid molecule can be provided separately or as a preformed complex.

In the cleavage assay, a nucleic acid molecule having a recognition sequence of the transposase/integrase is tagged with a fluorescent label (“fluorophore”) using conventional methods. The tagged molecule has a characteristic rotation measurable by FP. In general, larger molecules have higher FP values than smaller molecules as larger molecules tend to rotate more slowly than smaller molecules. The fundamentals of FP are not detailed here as they are well understood in the art. Reference is made to the examples below as well as Owicki, J. C., J. Biomolec. Screening 5:297, 2000 (incorporated herein by reference as if set forth in its entirety) for the technical details useful in establishing FP assays in accord with the invention.

It is noted that the fluorescent tag is provided on a “donor” end of the nucleic acid molecule that can be catalytically cleaved by the transposase/integrase such that a cleaved fragment carries the fluorescent tag. The full-length nucleic acid molecule can be a DNA molecule of between about 20 and 60 nucleotides and containing a transposase/integrase recognition sequence. When cleaved, the fluorescent fragment is preferably between about 1 and 20 nucleotides in length. It will be appreciated that shorter tagged molecules are preferred in the FP assay. A suitable fluorophore is selected to have a lifetime (i.e., the time between excitation and emission) that permits distinction between the larger protein/nucleic acid complex and smaller tagged nucleic acid molecules.

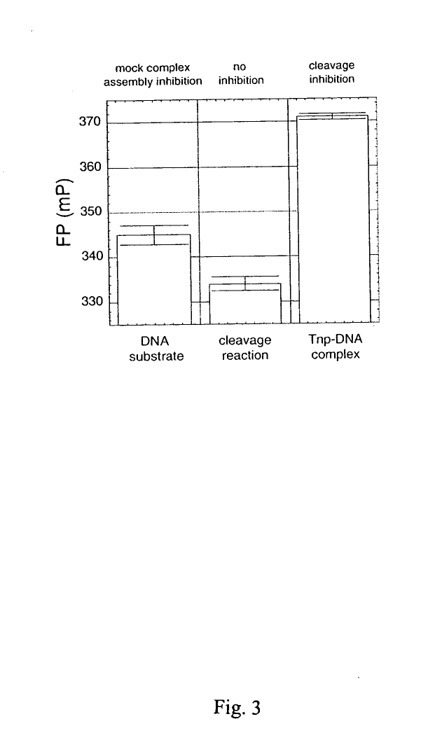

Under the conditions employed by the inventors, the FP of the tagged molecule is substantially the same as that of the fluorescent fragment generated by catalytic cleavage of the full-length nucleic acid molecule (FIGS. 1 and 3). On the other hand, when the tagged full-length nucleic acid molecule is complexed with a transposase/integrase protein, rotation of the molecule is reduced and FP is relatively higher than for the nucleic acid molecule itself (FIGS. 1 and 3). Accordingly, a skilled artisan can readily employ a suitable control to observe nucleic acid cleavage in the cleavage assay. One suitable control is a positive cleavage control that comprises the transposase/integrase protein and the nucleic acid molecule under the conditions that allow the transposase/integrase to bind and cleave the nucleic acid molecule. Another suitable control is a pseudo-positive cleavage control that consists essentially of the fluorescently labeled nucleic acid molecule (substrate) not bound by the transposase/integrase protein. In the above two cases, the FP of an experimental group in which the nucleic acid molecule has been cleaved will be comparable to the control FP. Another suitable control is a negative cleavage control that comprises the transposase/integrase protein and the nucleic acid molecule under the conditions that inhibit the nucleic acid cleavage activity of the transposase/integrase but allow the binding between the transposase/integrase and the nucleic acid molecule. In this case, the FP of the experimental group in which the nucleic acid molecule has been cleaved will be lower than the control FP. In a preferred embodiment, at least two controls, such as the positive and negative cleavage controls, are employed.

The transposase/integrase recognition sequences employed to practice the present invention can be those sequences known to the skilled artisan to support non-covalent binding of the transposase/integrase and are not limited to the natural recognition sequences. In the case of TnS transposase, for example, it will be appreciated that such sequences are not limited to the naturally occurring outside end termini (“OE termini”) or inside end termini (“IE termini”) of Tn5, but also extend to so-called mosaic termini described in the available patent and scientific literature, as well as other convenient variations of any of the foregoing. Reaction conditions for the members of the transposase/integrase superfamily are either known in the art or can be readily determined by a skilled artisan. For example, Tn5-based transposition systems useful in the invention, and related technologies, are described in U.S. Pat. Nos. 6,437,109, 6,406,896, 6,294,385, 6,159,736, 5,965,443, 5,948,622, and 5,925,545, each of which is incorporated herein by reference as if set forth in its entirety. In general, the nucleic acid cleavage activity of the members of the transposase/integrase superfamily requires the presence of divalent cation and the nucleic acid molecule can either be maintained in the form of a nucleic acid-transposase/integrase complex or cleaved to release a cleavage product by controlling the availability of divalent cations.

The cleavage assay of the present invention can be employed to screen for agents that can modulate the cleavage activity of a transposase/integrase. Such a screen includes the steps of:

a) incubating a transposase/integrase protein and a nucleic acid molecule that comprises a recognition sequence of the transposase/integrase under conditions that allow the transposase/integrase to bind, specifically and non-covalently, to the recognition sequence and cleave the nucleic acid molecule wherein the nucleic acid molecule is labeled with a fluorescent label at a position such that upon the cleavage a cleaved fragment not bound by the transposase/integrase and carries the fluorescent label is released;

b) exposing the transposase/integrase protein and the nucleic acid molecule of step a) to a test agent;

c) measuring the FP of the label either during or following step b); and

d) comparing the FP measurement of step c) to that of at least one control to determine whether the test agent can modulate the nucleic acid cleavage activity of the transposase/integrase.

For steps a) and b), the order in which the transposase/integrase, the nucleic acid molecule, and the test agent are added is not critical.

Examples of suitable controls include but are not limited to the positive cleavage control (e.g., a group run in parallel with the test agent group but not exposed to the test agent), the pseudo-positive cleavage control, and the negative cleavage control described above. If a test agent can inhibit the cleavage activity of the transposase/integrase, the FP value of the treated (by test agent) group will be higher than that of the positive and mock cleavage controls (preferably at least 150% of the positive and mock cleavage controls) and comparable to that of the negative cleavage control (preferably within 80% or 90% of the negative control).

If the test agent inhibits the binding of the transposase/integrase to the nucleic acid molecule but not the cleavage activity of the transposase/integrase, the FP value of the treated group will be comparable to that of the pseudo-positive and positive cleavage controls as FP of the full length nucleic acid molecule is substantially the same as that of the fluorescent fragment generated by catalytic cleavage of the full-length nucleic acid. Therefore, the present cleavage assay can identify compounds that inhibit cleavage but not binding. As existing binding inhibitor screening assays cannot distinguish specific from nonspecific inhibitors, binding inhibitors are less preferred than cleavage inhibitors as pharmaceutical candidates because they are more likely to have undesirable side effects by non-specifically inhibiting binding between a plurality of proteins and nucleic acids. In this regard, the present cleavage assay is advantageous in that it is able to identify compounds specific for cleavage.

If the test agent can enhance either the binding of the transposase/integrase to the nucleic acid molecule, the cleavage of the nucleic acid molecule, or both, the FP value of the treated group will be lower than that of the positive cleavage control (preferably less than 50% of the positive cleavage control). Such an enhancer can also be identified with a time course experiment in which the agent treated group will reach a lower level of FP faster than a control group not exposed to the agent.

In a preferred screening assay, at least two controls such as the positive and negative cleavage controls are employed.

The screening assay can be practiced in a high throughput manner in which at least 10, preferably at least 96, and most preferably at least 384 agents are screened simultaneously. In this case, the mean FP value from all treated groups can serve as a control as it will either be the same as or approach that of a positive cleavage control because most compounds in a random screen will not significantly modulate the activity of the transposase/integrase. In one embodiment, individual samples having an FP value at least about two, and preferably at least about three, standard deviations from the mean are selected for further evaluation.

Transposase/Integrase Nucleic Acid Binding Assay and Screening for Binding Modulators

In another aspect, the present invention relates to a method for observing the binding of a transposase/integrase and its corresponding recognition sequence. The method includes the steps of:

a) providing a transposase/integrase protein and a fluorescently labeled nucleic acid molecule that comprises a recognition sequence of the transposase/integrase under the conditions that allow the specific, non-covalent binding between the transposase/integrase protein and the recognition sequence but do not allow the transposase/integrase to cleave the nucleic acid molecule;

b) measuring the FP of the label during or following step b); and

c) comparing the FP measurement of step b) to that of at least one control to determine the binding between the transposase/integrase and its recognition sequence.

For step a), the transposase/integrase protein and the nucleic acid molecule can be provided separately or as a preformed complex.

In comparison to the cleavage assay, the binding assay is based on the FP difference between a fluorescently labeled nucleic acid molecule and that of the nucleic acid-transposase/integrase complex. Therefore, the particular position or positions at which the nucleic acid molecule is labeled is not critical. In one embodiment, the nucleic acid molecule is a DNA molecule of between about 20 and 60 nucleotides and containing a transposase/integrase recognition sequence. As in the transposase/integrase cleavage assay, the transposase/integrase recognition sequences employed in the binding assay can be those sequences known to the skilled artisan to support non-covalent binding of the transposase/integrase and are not limited to the natural recognition sequences.

One suitable control for the binding assay is a positive binding control that comprises the transposase/integrase protein and the nucleic acid molecule under the conditions that allow the transposase/integrase to bind to but do not allow the transposase/integrase to cleave the nucleic acid molecule. In this case, the FP value of an experimental group will be comparable to that of the control. Another suitable control is a negative binding control that consists essentially of the fluorescently labeled nucleic acid molecule not bound by the transposase/integrase protein. In this case, the FP value of the experimental group will be higher than that of the control.

In a preferred embodiment of the binding assay, both controls are employed.

The above binding assay can be used to screen for agents that can modulate the binding between the transposase/integrase and the nucleic acid molecule. The screening involves:

a) providing a transposase/integrase protein and a fluorescently labeled nucleic acid molecule that comprises a recognition sequence of the transposase/integrase under the conditions that allow the specific, non-covalent binding between the transposase/integrase protein and the recognition sequence but do not allow the transposase/integrase to cleave the nucleic acid molecule;

b) exposing the transposase/integrase protein and the nucleic acid molecule to a test agent;

c) measuring the FP of the label during or following step b); and

d) comparing the FP measurement of step c) to that of at least one control to determine whether the agent can modulate the binding between the transposase/integrase and its recognition sequence.

For steps a) and b), the order in which the transposase/integrase, the nucleic acid molecule, and the test agent are added is not critical.

Examples of suitable controls for the screening assay include but are not limited to the positive and negative binding controls described above. If the test agent can inhibit the binding between the transposase/integrase and the nucleic acid molecule, the FP of the treated group will be lower than that of the positive control (preferably less than 50% of the positive control) and comparable to that of the negative control (preferably with 80% or 90% of the negative control). If the test agent can enhance the binding between the transposase/integrase and the nucleic acid molecule, the FP of the treated group will be higher than that of the positive control (preferably at least 150% of the positive control). Such an enhancer can also be identified with a time course experiment in which the test agent treated group will reach a higher FP level faster than a control group not exposed to the agent. In one embodiment, a candidate inhibitor is of interest if it has an FP value significantly lower than, and preferably no more than about 50% of, the positive binding control and a candidate enhancer is of interest if it has an FP value significantly higher than, preferably more than 150% of, the positive binding control.

In a preferred screening assay, both the positive and negative binding controls are employed.

The screening assay can be practiced in a high throughput manner in which at least 10, preferably at least 96, and most preferably at least 384 agents are screened simultaneously. In this case, the average FP from all treated groups can serve as a control as it will either be the same as or approach that of a positive binding control because most compounds will not significantly modulate the binding between the transposase/integrase and the nucleic acid molecule. In one embodiment, individual samples having an FP value at least about two, and preferably at least about three, standard deviations from the mean are selected for further evaluation.

Compounds that Inhibit Tn5 Transposase and HIV Integration

It is disclosed here that compounds defined by the following formula I have anti-HIV activities and can be used to inhibit HIV integration in cells by exposing target HIV viruses to one or more these compounds: 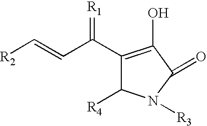
wherein R₁ is O or S and R₂ to R₄ are identical or different and represent an aryl group such as a phenyl group or a substituted aryl group such as a substituted phenyl group.

In a preferred embodiment, R₁ is O.

In another preferred embodiment, R₂-R₄ (can be the same or different) are represented by 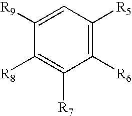
wherein R₅ to R₉ are identical or different and are selected from the group consisting of a hydrogen atom, a hydroxyl group, a carboxyl group, a halogen atom, a nitro group, C≡N, an amino group, S—H, and a carbon chain of 1-6 carbons, the carbon chain can be saturated, unsaturated, linear, or branched and can contain have heteroatoms attached as part of the chain or a side group wherein the heteroatoms are selected from the group consisting of F, Cl, Br, I, O, S, P, and N.

In another preferred embodiment, R₂ is a phenyl group, R₃ is selected from the group consisting of a phenyl group, a C₁₋₃ alkoxy group substituted phenyl group, and a trifluoro group substituted phenyl group, and R₄ is a nitro group substituted phenyl group.

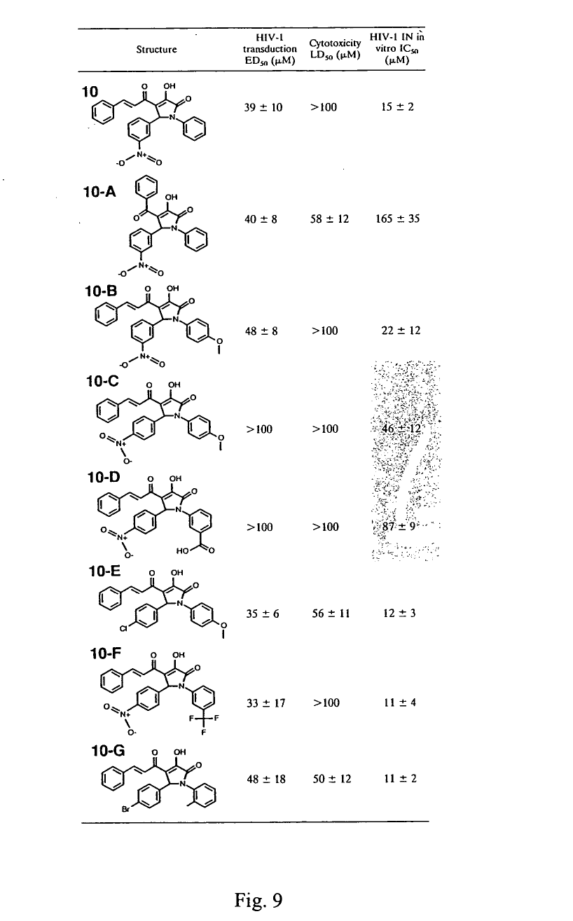

In still another preferred embodiment, the HIV integration inhibitor is selected from compound 10 (FIG. 9), compound 10B (FIG. 9), compound 10F (FIG. 9), and a compound having the formula

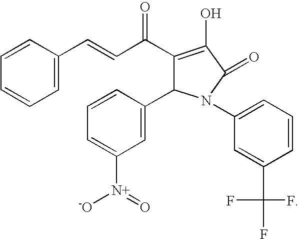

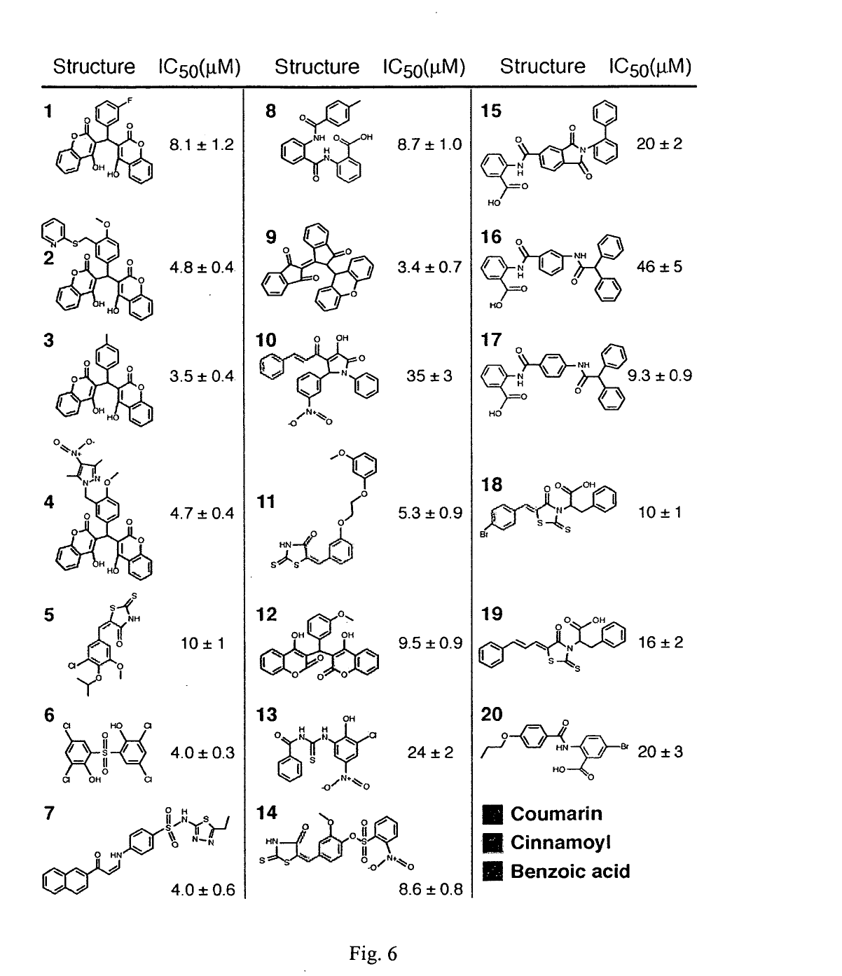

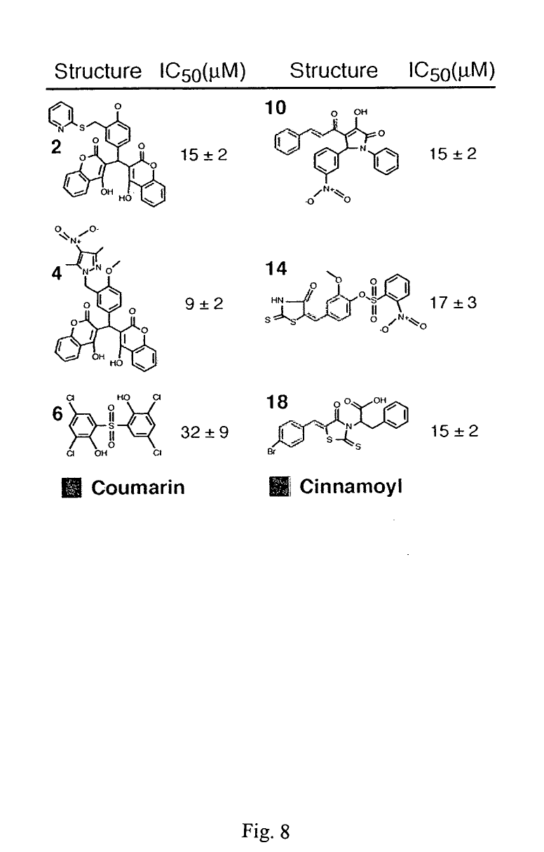

The determination of the above class of compounds as HIV integration inhibitors started with a high throughput screen for Tn5 transposase inhibitors followed by further testing, characterization, and structure activity study of candidate compounds (identified from the screen) using various Tn5 transposase and HIV integrase and integration systems (example 2). Twenty Tn5 transposase inhibitors (FIG. 6 in example 2) and 12 HIV integrase inhibitors (FIGS. 8 and 9 in example 2) were also identified during this process (example 2). The Tn5 transposase and HIV integrase inhibitors can be used to inhibit the activity of these enzymes by exposing the enzymes to the inhibitors. It is noted that the task of further evaluating the compounds identified from the screening assay is appreciably more technically feasible and approachable than undertaking a general analysis of the 16,000 compounds screened in the assay without prior selection.

The transposase/integrase assays of the present invention as well as the Tn5 transposase and HIV integration inhibitors identified by the assays are further illustrated in the examples below in connection with a Tn5-based system, which is a preferred embodiment of the present invention, especially for identifying candidate HIV integration inhibitors. In particular, the examples illustrate methods of identifying molecules that inhibit the Tn5 transposase-mediated strand cleavage and identifying molecules that inhibit binding between the Tn5 transposase and the transposase recognition sequences in the transposable nucleic acid. The examples compared these Tn5-based methods to prior methods for assessing binding and cleavage, with consistent outcomes. In keeping with the general methods described herein, it is understood that the invention can be practiced with other proteins of the transposase/integrase superfamily by using other proteins with their corresponding recognition sequences as well as their corresponding binding and cleavage conditions, which are either known in the art or can be readily determined by a skilled artisan.

The present invention is not intended to be limited by the examples below. Rather the invention is understood to encompass all the variations and modifications that come within the scope of the appended claims.

### EXAMPLE 1

### A High-Throughput Assay for Tn5 Transposase Induced DNA Cleavage

Materials and Methods

DNA substrates: The short oligonucleotides used for these experiments are purchased from Integrated DNA Technology (IDT). The short oligonucleotides are annealed to form double stranded DNA (dsDNA) by adding two μmoles of each oligonucleotide to a 20 mM Tris-HCl pH 7.9, 10 mM NaCl solution for a 2 μM final oligonucleotide concentration. To anneal the single stranded DNA (ssDNA), the oligonucleotides are heated at 96° C. for one minute followed by a decrease in temperature at 0.1° C. per second to 4° C. The transferred strand is labeled with rhodamine green for the FP assays or fluorescein for native gel-shift assays. Fluorescent oligonucleotides were purchased high-performance liquid chromatography (HPLC) purified from IDT.

The sequence of the 50 nt DNA fragments used for FP assays are 5′ TGC AGG TCG ACT GTC TCT TAT ACA CAT CTT GAG TGA GTG AGC ATG CAT GT 3′ (SEQ ID NO:1) and its complement. The dsDNA produced from these two fragments consists of 10 bp of donor and 40 bp of transposon DNA. Only the 5′ end of the non-transferred strand is fluorescently labeled for the experiments using this dsDNA substrate. The sequence of the 60 nt DNA fragment used for the gel-shift assays are 5′ GGC CAC GAC ACG CTC CCG CGC TGT CTC TTA TAC ACA TCT TGA GTG AGT GAG CAT GCA TGT 3′ (SEQ ID NO:2) and its complement. The dsDNA produced from these two fragments consists of 20 bp of donor and 40 bp of transposon DNA, which are both labeled with fluorescein on their 5′ ends.

Transposase purification: The EK54, MA56 and LP372 hyperactive mutant version of transposase is used for all assays and will be referred to as transposase throughout this manuscript. Transposase is purified as described previously (Ason, B. and Reznikoff, W. S., J Biol Chem 277: 11284-11291, 2002). All transposase protein preparations are quantitated using a Bradford assay with Bovine Serum Albumin (BSA) as the standard.

Strand cleavage assays: In these assays, two DNA fragments each containing the transposase recognition sequence are used to mimic the Tn5 transposon. The cleavage reactions are carried out by incubating 800 nM transposase with 160 nM dsDNA at 37° C. for 1.5 hours in cleavage buffer (25 mM HEPES pH 7.5, 2 mM Tris-HCl pH 7.5, 100 mM potassium glutamate, 9 mM NaCl, 0.5 mM β-mercaptoethanol, 10 μg/mL t-RNA, 0.25 mg/mL BSA, 9% glycerol, and 10 mM MgAc). For FP analysis, the 60 μL reactions are analyzed using the FP protocol on a Wallac Victor V plate reader with the instruments fluorescein filters, F485 excitation and F535 emission. The readings are taken 8 mm from the bottom of the plate with the G factor set at 1 and a 0.1 sec counting time. The polarization aperture is set at ‘normal’ and the CW-lamp energy is set at the maximum, 65 535.

For gel shift assays, following the 37° C. incubation, a 20 μL aliquot of each reaction is mixed with 6 μL of 6× loading dye (Promega) and electrophoresed on either a 9% or 10% native polyacrylamide gel at 300V. After 3 hours, the gel is scanned using a Fluorlmager SI (Vistra Fluorescence), and the bands are quantitated using Image Quant (Molecular Dynamics). Increasing concentrations of EDTA (0, 0.01, 0.05, 0.1, 0.5, 1, 2, 3, 4, 5, 10, 20, 50 mM) are used as a metal chelator during assay development, since strand cleavage is metal dependent.

Results

Development of a high-throughput screen for Tn5 transposase induced strand cleavage: This assay was developed to screen for small molecules that specifically inhibit transposase cleavage activity. Our method is based on the change in polarization of a fluorescently tagged dsDNA fragment. In general, FP measures the tumbling rate of a population of fluorescently labeled molecules between the time of fluorophore excitation and emission. For this assay, we used short fluorescently labeled dsDNA fragments each containing one transposase recognition sequence. These dsDNA fragments are selectively labeled on the donor DNA end, the 5′ end of the non-transferred strand (FIG. 1A). Following strand cleavage, the donor fragment is released. Thus, as the population of cleaved DNA fragments increases, the FP value decreases, since the shorter labeled DNA fragments exhibit a faster tumbling rate in solution (FIG. 1B).

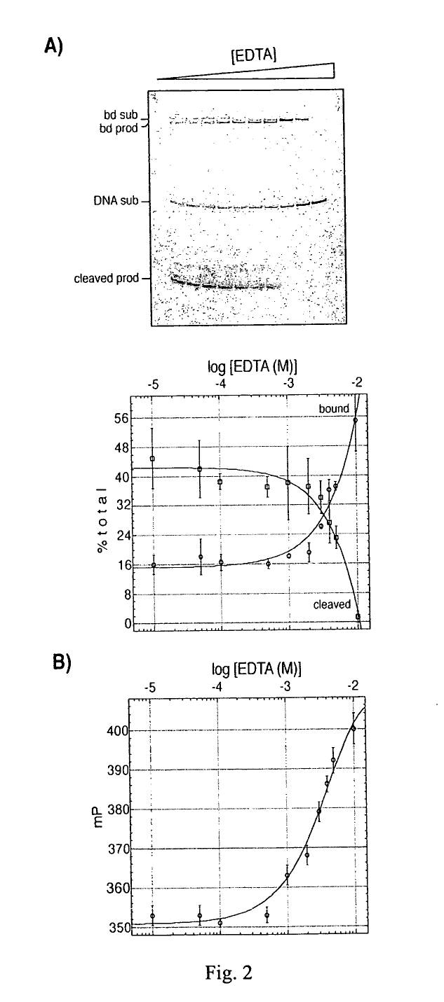

A comparison of these FP and gel shift assay data reveal that the two assays are in good agreement with one another (FIG. 2). In these experiments, increasing concentrations of EDTA were added to the reactions to inhibit catalysis. Thus, the change in FP represents a shift in the population towards more transposase-DNA complexes compared to free cleaved DNA fragments in solution. In the gel shift assays, inhibition of strand cleavage corresponds to an increase in signal for the band corresponding to transposase-DNA complexes paralleled by a decrease in the band corresponding to the cleaved DNA product. Comparing FIGS. 2A and B reveals that the same concentration of EDTA inhibits strand cleavage in both the FP and gel shift assays suggesting that under these conditions FP is a good measurement of the degree of strand cleavage. Furthermore, this FP assay has a z′ value of 0.57, under these conditions, indicating that the assay window and precision are sufficient for high-throughput screening (Zhang, J. H. et al., J Biomol Screen 4: 67-73, 1999).

It is worth noting that the order of addition of the DNA, transposase, and potential inhibitor is not critical for distinguishing between inhibitors of complex assembly and cleavage. Instead, one simply needs to compare each test reaction to the average value obtained for the entire screen, which eliminates the need to take multiple measurements per sample test reaction. The average sample value typically lies near the value for an uninhibited reaction, since most compounds in a random screen would not have an effect on the reaction. In our case, this would be the transposase induced DNA cleavage reaction, which was verified through the addition of the proper controls such as the uninhibited cleavage reaction and transposase-DNA complexes unable to undergo cleavage (mock-inhibited reactions).

Applications: Here we present the development of a high-throughput screen to identify compounds that inhibit TnS transposase induced strand cleavage. This technique will be useful for identifying compounds that specifically inhibit catalysis, since the free DNA substrate and the cleaved donor DNA tumble relatively quickly compared to the transposase-DNA complex (FIG. 3). Thus, compounds that directly inhibit catalysis can be distinguished from inhibitors of complex assembly. This method, in effect, increases the specificity for strand cleavage and reduces the population of hits produced from library screening, since any compound that inhibits complex assembly would exhibit a similar FP value to the uninhibited reaction. The elimination of compounds that inhibit transposase-DNA binding is advantageous, since this should reduce the number of promiscuous inhibitors that are identified as hits, focusing the screen on compounds that specifically inhibit catalysis.

Compounds identified from this screen could be used to trap intermediates of the reaction. Previous data suggest that conformational differences exist between different transposase-DNA intermediates as well as metal bound and free complexes for this protein superfamily (Steiniger-White, M. et al., J Mol Biol 322: 971-982, 2002; Asante-Appiah, E. et al., J Biol Chem 273: 35078-35087, 1998; Asante-Appiah, E. et al., Adv Virus Res 52: 351-369, 1999; Ciubotaru, M. et al., J Biol Chem 278: 5584-5596, 2003; Williams, T. L. and Baker, T. A., J Biol Chem 279: 5135-5145, 2004; Allingham, J. S. and Haniford, D. B., J Mol Biol 319: 53-65, 2002; Mundy, C. L. et al., Mol Cell Biol 22: 69-77, 2002; and Hwang, Y. et al., Nucleic Acids Res 28: 4884-4892, 2000). Effectors identified using this screen would be particularly useful in examining metal bound cleavage intermediates, which would otherwise be difficult to trap. Co-crystallization studies with any compound that affects cleavage in a transposase-DNA-compound structure could identify the structural basis for the interaction, and one could imagine using these structures in a rational drug design approach, modeling the necessary chemical augmentations required to fit the active site of other superfamily members, such as HIV-1 integrase.

Furthermore, since the transposase/integrase superfamily shares high structural similarities within their catalytic cores, compounds that inhibit Tn5 transposase may cross react with other family members (Dyda, F. et al., Science 266: 1981-1986, 1994; Bujacz, G. et al., J Mol Biol 253: 333-346, 1995; Rice, P. and Mizuuchi, K., Cell 82: 209-220, 1995; and Davies, D. R. et al., Science 289: 77-85, 2000). Thus, it is likely that compounds identified as cleavage inhibitors of Tn5 transposase could be useful both as mechanistic probes and in drug discovery for these other family members.

Comparison to other high-throughput assays: Most high-throughput assays targeting this superfamily have focused on HIV-1 integrase (Hazuda, D. J. et al., Science 287: 646-650, 2000; Hazuda, D. J. et al., Nucleic Acids Res 22: 1121-1122, 1994; Craigie, R. et al., Nucleic Acids Res 19: 2729-2734, 1991; Vink, C. et al., Nucleic Acids Res 22: 2176-2177, 1994; Hazuda, D. et al., Drug Des Discov 15: 17-24, 1997; and Hazuda, D. J. et al., J Virol 71: 7005-7011, 1997). These assays typically monitor the incorporation of a labeled substrate (the donor) into an immobilized target or the attachment of a labeled target to an immobilized donor. The read-out for these screens focuses on the covalent attachment, or integration, of the donor to the target. Therefore, inhibition could occur at any step of the reaction such as assembly, 3′ end processing, or strand transfer. Thus, it is difficult to distinguish between catalytic and complex assembly inhibitors using these assays. One approach to address this involves pre-forming complexes prior to library screening. This has been effective at distinguishing between inhibitors of complex assembly and catalysis in several instances (Hazuda, D. J. et al., J Virol 71: 7005-7011, 1997; and Owicki, J. C., J Biomol Screen 5: 297-306, 2000). However, it remains likely that an inhibitor could disrupt a pre-assembled complex upon addition to the reaction, and since these assays do not measure complex assembly directly, this would be difficult to detect.

The potential drawback for any fluorescence assay used in compound screening is the occurrence of false positives from intrinsically fluorescent compounds. However, most fluorescent compounds can be readily identified as fluorescent, since the signal for reactions containing fluorescent compounds are typically well outside the assay window for our screen. Furthermore, confirming a compound's intrinsic fluorescence can easily be determined by monitoring the fluorescence of the compound alone.

### EXAMPLE 2

### Targeting Tn5 Transposase Identifies HIV-1 Inhibitors

Materials and Methods

Compounds: Compound screening was performed at the University of Wisconsin-Madison Comprehensive Cancer Center Small Molecule Screening Facility. This library was originally purchased from ChemBridge. Compound numbers in this example correspond to the following ChemBridge ID numbers: 1=6160027, 2=6141194, 3=6140731, 4=6158572, 5=5868253, 6=6075259, 7=5980789, 8=6058083, 9=6229546, 10=6176494, 11=6192779, 12=5546355, 13=5535396, 14=6227564, 15=5233170, 16=5232986, 17=5232985, 18=6046791, 19=6044999, 20=5988232, 10-A=5789176, 10-B=6204337, 10-C=6206397, 10-D=8065508, 10-E=6171674, 10-F=6180772, 10-G=6215673.

DNA substrates: The oligonucleotides were purchased HPLC purified from IDT. dsDNA was formed by adding two μmoles of each oligonucleotide to 10 mM Tris-HCl pH 7.9 and 10 mM NaCl. The oligonucleotides were either heated to 96° C. for one minute followed by a decrease in temperature at 0.1° C./sec to 4° C. or heated to 90° C. in a 2 L water bath and cooled to 8° C. overnight. The transferred strand was 5′ end labeled with either rhodamine green for FP or fluorescein for gel shift assays.

The 19 bp DNA sequences used for FP assays were 5′ C TGT CTC TTA TAC ACA TCT 3′ (SEQ ID NO:3) and its complement. The 40 bp DNA sequences used for the gel shift assays during assay development were 5′ C TGT CTC TTA TAC ACA TCT TGA GTG AGT GAG CAT GCA TGT 3′ (SEQ ID NO:4) and its complement. The 60 bp DNA sequences used for verification of inhibition were 5′ GGC CAC GAC ACG CTC CCG CGC TGT CTC TTA TAC ACA TCT TGA GTG AGT GAG CAT GCA TGT 3′ (SEQ ID NO:5) and its complement.

Transposase and integrase purification: The EK54, MA56 and LP372 hyperactive mutant version of transposase was used for all assays and is referred to as transposase throughout this work. Transposase and integrase proteins were purified as described in Ason, B., and W. S. Reznikoff, J Mol Biol 335:1213-25, 2004 and Taganov, K. D. et al., Journal of Virology 78:5848-55, 2004, both are herein incorporated by reference in their entirety.

Viruses and cell lines: 293T cells were obtained from the ATCC. Ghost-R5 (Morner, A. et al., J Virol 73:2343-9, 1999) cells were obtained from the NIH AIDS Reagent Repository. HIV-1 single cycle reporter viruses were produced by cotransfection of 293T cells with pNL4-3.Luc.R-E-(Connor, R. I. et al., Virology 206:935-44, 1995; and He, J. et al., J Virol 69:6705-11, 1995), and HIV-1 Env expression vector pSV7d-JR.FL (Deng, H. et al., Nature 381:661-6, 1996).

FP and gel-shift assays: Reactions were performed as described in Ason, B., and W. S. Reznikoff, J Mol Biol 335:1213-25, 2004, except 800 nM transposase and 160 nM dsDNA were used for complex formation. For FP analysis, 60 μL reactions were analyzed using the FP protocol on a Wallac Victor V plate reader and a 7% native polyacrylamide gel was used in gel shift assays. For compound screening and subsequent analysis, 1 μL of either DMSO or test compound (final concentration 80 μM) was added to the synapsis reactions. Compound screening was carried out using a Beckman Coulter Biomek FX in a 384 well format. Compounds identified as hits were rescreened using gel shift assays under synapsis conditions with a 120 μM compound concentration. IC₅₀ values were obtained by fitting inhibitor titration data (0, 0.01, 0.05, 0.1, 0.5, 2.5, 10, 20, 35, 50, 100, 200, 400, 800 μM) to an exponential decay.

Inhibition of the restriction enzyme BsmA I was analyzed to determine compound specificity. Reactions were carried out in 2X NEB Buffer 3 with 160 nM dsDNA, 10 units of BsmA I (New England Biolabs), and 120 μM lead compound. Reactions were incubated at 55° C. for one hour followed by a 20 minute 80° C. heat inactivation step prior to gel electrophoresis. Samples were loaded onto a 9% native polyacrylamide gel and run at 300V for 2.5 hours. Gels were subsequently imaged and quantitated as described for the synapsis assays.

HIV-1 integrase assay: HIV-1 integrase activity was measured as described in Daniel, R. et al., AIDS Res Hum Retroviruses 20:135-44, 2004 (incorporated by reference in its entirety), except that 1.0 μL of either DMSO or inhibitor were added to the reactions. Briefly, HIV-1 integrase (1 μM, final concentration) was pre-incubated with various concentrations of inhibitor at 30° C. for 30 min. A 21-base pair ³²p labeled substrate (1×10⁶ dpm), representing the U5 end of the viral genome, and MnCl₂ (10 mM, final concentration) were subsequently added to the reaction. The reaction proceeded for 15 min. at 37° C. The reactions were subsequently quenched using EDTA (10 mM, final concentration), and the products separated on a 20% denaturing polyacrylamide gel.

HIV-1 transduction assay and inhibitory screen: 10,000 Ghost-R5 cells per well were plated in a 384 well plate in 49 μL media. 18 hours after plating, 1 μL of inhibitory compound, dissolved in DMSO, was added and gently mixed into solution. Following a 1 hour 37° C. incubation, HIV-1 virions were added in a final infectious volume of 100 μL. 44-48 hours after infection, 87 μL of media was removed from each well, and 13 μL/well of Bright-Glo luciferase detection reagent (Promega) was added. After a 2 minute incubation, the plates were read on a multiwell plate luminometer.

Cytotoxicity assay: Cytotoxicity assays were performed exactly as the infection assay described above, except media without virus was added following compound addition, and Cell Titer Glo (Promega) viability reagent was added in place of Bright Glo.

Quantitative real-time PCR of reverse transcripts, detection of transduction in newly infected cells, and effect of compounds on established provirus expression in stably infected cells: 10,000 Ghost-R5 cells were plated in 96 well dishes, treated with compound in a total volume of 75 μL, and incubated at 37° C. for 1 hour. Virus was then added in an additional volume of 25 μL. The final infectious volume was 100 μL, the final DMSO concentration was 1%, and the final compound concentration was as indicated. DNA was harvested using the DNeasy kit (Qiagen) 24 hours after infection in the constant presence of inhibitory compounds. Late DNA products of HIV reverse transcription were quantitated using the primers MH531 and MH532, and the probe LTR-P, as described in Burke, T. R. et al., J Med Chem 38:4171-8, 1995, herein incorporated by reference in its entirety.

In parallel, cells infected with virus and compound were assayed for luciferase expression by the addition of 100 μL of Bright-Glo reagent, as described above. In this way, effect of the inhibitory compounds on luciferase expression brought about by successful transduction was measured in the constant presence of the compounds.

To assess the potential effects of the inhibitory compounds on post-integration events such as transcription, translation, and luciferase enzyme stability and activity, Ghost-R5 cells stably infected several weeks earlier with the same virus preparation were incubated with inhibitory compound in 96 well dishes and luciferase activity was monitored 48 hours later by the addition of 100 μL of Bright-Glo reagent to each well, as described above.

Results

Identification of Tn5 transposase inhibitors: We developed a FP based, transposase-DNA complex assembly assay to screen small molecule libraries for inhibition of transposase-DNA complex formation. In our assay, we monitored the change in polarization of a fluorescently labeled short DNA fragment containing one transposase recognition sequence. In general, FP measures the tumbling rate, due to Brownian motion, of a population of fluorescently labeled molecules in solution between the time of fluorophore excitation and emission. Thus, when measuring transposase-DNA binding interactions, if the percentage of DNA bound by the transposase increases, the population of more slowly rotating transposase bound DNA fragments increases, thereby increasing the FP value.

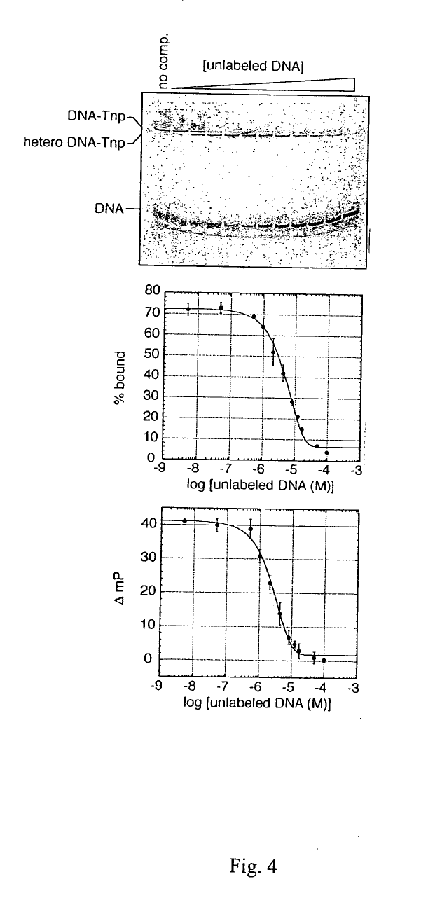

Comparison of the data obtained using either FP or gel shift assays to monitor transposase-DNA binding interactions during synapsis reveals that the two assays are in good agreement with one another (FIG. 4). In these experiments, increasing concentrations of an unlabeled DNA was added to the reaction to serve as a competitive inhibitor to transposase-labeled DNA interactions. These experiments reveal that in each assay the unlabeled DNA reduces the fraction of labeled DNA bound by transposase within the same concentration range. The observed IC₅₀ value is 2.6±0.2 μM and 5.1±0.4 μM for data obtained from either the FP or gel shift assays respectively.

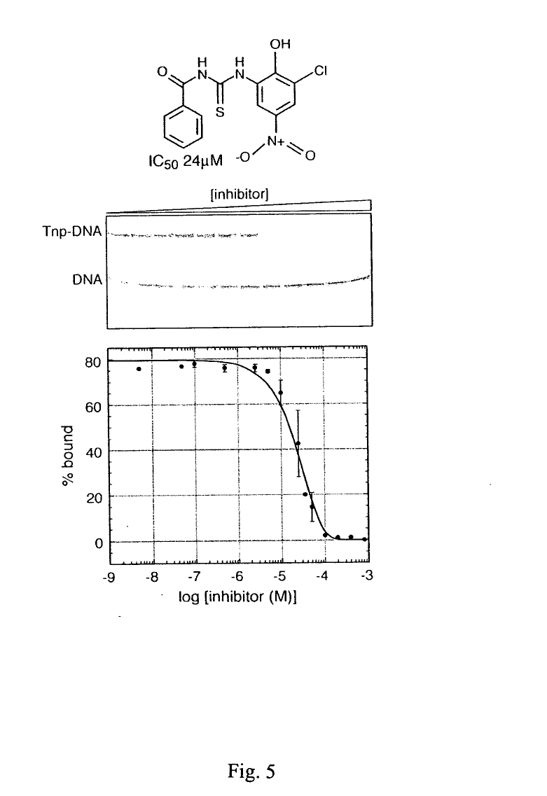

During this initial application, we screened 16,000 pharmacologically active compounds or their derivatives. From this library, 76 compounds were identified as effectors of transposase-DNA complex assembly. Of these, 20 were identified as inhibitors in several unrelated assays and were therefore considered promiscuous and ignored. From the remaining compounds, 39 were verified to inhibit complex assembly, as observed by gel shift assays (FIG. 5). To further narrow the number of lead candidates and to focus our search to compounds that were specific to the transposase/integrase superfamily, we tested whether these compounds also inhibited the restriction enzyme BsmA I. This type II restriction enzyme recognizes a site within the transposase recognition sequence. Any compound that inhibits BsmA I activity is thus classified as low specificity tranposase inhibitor. Of these 39 compounds, 20 compounds did not significantly inhibit BsmA I activity.

The 20 compounds that selectively inhibit Tn5 transposase are largely aromatic, which is representative of the library as a whole, and exhibit IC₅₀ values ranging from 3.5 to 46 μM (FIGS. 5 and 6). Within this group, several subsets of compounds appear to be structurally related. One group consists of five coumarin dimers (compounds 1-4 and 12). Another consists of benzoic acid derivatives (compounds 8, 15-17, and 20). The last group (compounds 5, 10, 11, 14, 18, and 19) contains various conformations of a cinnamoyl moiety. The remaining compounds appear to be unique.

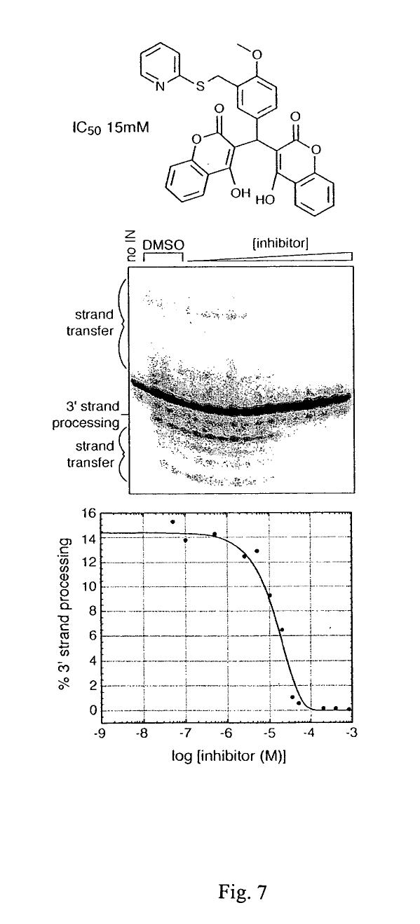

In vitro inhibition of HIV-1 integrase: We found that six of the 20 compounds that selectively inhibit Tn5 transposase also significantly inhibited the activity of HIV-1 integrase, as observed by PAGE analysis of the reaction products from in vitro integration (FIGS. 7 and 8). In an assay containing 1 μM of HIV integrase, the IC₅₀ values for these compounds range from 9 to 32 μM. In all cases, integrase inhibition is marked by a parallel decrease in the products of both the 3′ strand processing and strand transfer reactions. Inhibition constants were therefore calculated exclusively from the inhibition of 3′ strand processing, as these data were more quantifiable. These inhibitors can be classified into three types of structures, coumarin dimers (compounds 2 and 4), cinnamoyl derivatives (compound 10, 14, 18), and a chlorinated bithionol sulfoxide (compound 6).

Inhibition of HIV-1 in cells: These compounds were tested further to determine if they were effective at blocking HIV transduction (a readout for successful integration) in the absence of cytotoxicity. Compound 10, a cinnamoyl derivative, inhibits transduction with an ED₅₀ of 39 μM and at least 2-fold greater LD₅₀ (FIG. 9). In a structure activity study, compound 10 derivatives 10-A through 10-G were tested for their effects on integrase in vitro activity, HIV-1 transduction, and cellular toxicity. This study suggest that the both the ethylene linker within the cinnamoyl moiety and two functional groups located off of the central pyrrole and away from the cinnamoyl play a role in both compound efficacy and toxicity.

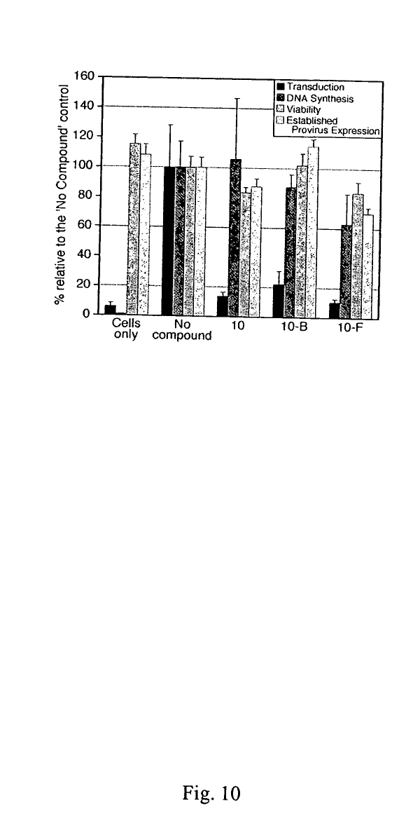

Three compounds (10, 10-B, and 10-F) were further tested for their effects on events in the viral lifecycle both upstream and downstream of integration. Virus reverse transcripts were quantitated by real-time PCR. Under conditions in which transduction was inhibited by 75% or greater, viral DNA synthesis was either uninhibited, or only inhibited to a small extent, suggesting that events up to and including reverse transcription were not affected by the compounds (FIG. 10). In order to test the effect of the compounds on events after integration, including transcription and translation, and to control for effects on luciferase enzyme stability and activity, cells chronically infected with the same luciferase-encoding HIV-1 virus were assayed in parallel. As shown in FIG. 10, the inhibitory compounds had no effect on luciferase expression in these cells. Finally, the compounds were found to have no significant effect on gross cell viability, as determined using a traditional assay for cellular metabolic activity (FIG. 10). Together, the inhibitory effect of these compounds on Tn5 transposase and HIV-1 integrase in vitro coupled with their effect in vivo to a point in the viral lifecycle post reverse-transcription, but upstream of transcription from the provirus, suggest that the compounds are inhibiting retroviral integration.

Discussion: This example provides evidence to support the use of Tn5 transposase as a surrogate model for HIV-1 integrase inhibitor development. Six compounds were identified that inhibit the activities of both Tn5 transposase and HIV-1 integrase, yet they do not inhibit the restriction enzyme Bsm A1. In addition, these compounds were not identified as hits in any other screen used at the facility, including other FP assays, and it should be noted that for compounds tested, inhibition of transposase synapsis reactions were not affected by the addition of an excess of unlabeled plasmid to chase away any potential inhibition due to nonspecific DNA-compound interactions. This indicates that these compounds are not interacting with the DNA but with a region along the protein conserved between transposase and integrase and not Bsm A1.

These compounds were originally identified as inhibitors of transposase complex assembly and are likely inhibiting integrase-DNA interactions as well, providing evidence that both coumarins and cinnamoyl integrase inhibitors target this step of the integration mechanism. It has been suggested that integrase inhibitors, which target complex assembly are undesirable, because several compounds found to inhibit integrase-DNA interactions were shown to be ineffective at inhibiting viral preintegration complexes (Farnet, C. M. et al., Proc Natl Acad Sci USA 93:9742-7, 1996). However, we identified three compounds that inhibit integrase in vitro, which also inhibit transposase assembly and HIV-1 infection in cells at a point in the viral lifecycle consistent with inhibition of integration (compounds 10, 10-B, 10-F).

Disruption of the cinnamoyl moiety through the removal of the ethylene group (FIG. 9, compound 10-A) increases cytoxicity and impedes its efficacy as an integrase inhibitor, suggesting that this moiety is important for inhibition. Interestingly, two reactive groups, a ketone and an adjacent enol form a diketo-like motif within the central pyrrole. This is reminiscent of the diketo moiety found in diketo acids, another extensively described class of integrase inhibitors (Hazuda, D. J. et al., Science 287:646-50, 2000). This diketo-like motif is also found in 5ClTEP. In fact, the 5ClTEP-IN co-crystal structure revealed that this moiety forms a hydrogen bond with E152, of the integrase DDE motif (Goldgur, Y. et al., Proc Natl Acad Sci USA 96:13040-3, 1999). Thus, the activity we observe could stem, in part, from this diketo-like moiety.

However, it is likely that neither the cinnamoyl nor the central pyrrole are the exclusive pharmacophores for these compounds, because two additional groups attached to the central pyrrole also have an impact on inhibition (FIG. 9). Furthermore, it is worth noting that compound 10-F partially inhibits BsmA 1 activity, suggesting that although this compound appears to inhibit HIV transduction, it may lack the desired specificity, a phenomenon previously reported for some cinnamoyl derivatives (Pluymers, W. et al., Mol Pharmacol 58:641-8, 2000).

In summary, the success of using Tn5 transposase as a surrogate for finding HIV-1 inhibitors suggests that similar surrogates can be used for other protein superfamilies. This would facilitate the use of simpler screens and the use of the best available structural data for inhibitor screening and development. This also alerts one to the possibility of undesirable cross activity with other members of the same superfamily of proteins. In this case, such cross reactivity could occur with the RAG proteins. These proteins are involved in DNA cleavage during immunoglobulin gene formation and share a similar catalytic mechanism, and presumably structure, to both transposase and retroviral integrases (van Gent, D. C. et al., Science 271:1592-4, 1996).

We claim:

1. A method for observing the nucleic acid cleavage activity of a member of the transposase/integrase superfamily, the method comprising the steps of:
a) incubating a transposase or integrase protein and a nucleic acid molecule that comprises a recognition sequence of the transposase or integrase under conditions that allow the transposase or integrase to bind, specifically and non-covalently, to the recognition sequence and cleave the nucleic acid molecule wherein the nucleic acid molecule is labeled with a fluorescent label at a position such that upon the cleavage a cleaved fragment not bound by the transposase or integrase and carries the fluorescent label is released;
b) measuring the fluorescence polarization of the label either during or following step a); and
c) comparing the fluorescence polarization measurement of step b) to that of at least one control to determine the cleavage of the nucleic acid molecule.

2. The method of claim 1, wherein the method is for observing the nucleic acid cleavage activity of a transposase.

3. The method of claim 2, wherein the transposase is selected from the group consisting of Tn5 transposase, Tn10 transposase, and Mu transposase.

4. The method of claim 3, wherein the transposase is Tn5 transposase.

5. The method of claim 1, wherein the nucleic acid molecule is a DNA molecule between 2 and 60 nucleotides in length.

6. The method of claim 1, wherein the control is selected from the group consisting of a positive cleavage control that comprises the transposase or integrase protein and the nucleic acid molecule under the conditions that allow the transposase or integrase to bind and cleave the nucleic acid molecule, a negative cleavage control that comprises the transposase or integrase protein and the nucleic acid molecule under the conditions that inhibit the nucleic acid cleavage activity of the transposase or integrase but allow the binding between the transposase or integrase and the nucleic acid molecule, and a pseudo-positive cleavage control that consists essentially of the fluorescently labeled nucleic acid molecule not bound by the transposase or integrase protein.

7. The method of claim 6, wherein at least two controls are employed.

8. A method for identifying an agent that can modulate the nucleic acid cleavage activity of a member of the transposase/integrase superfamily, the method comprising the steps of:
a) incubating a transposase or integrase protein and a nucleic acid molecule that comprises a recognition sequence of the transposase or integrase under conditions that allow the transposase or integrase to bind, specifically and non-covalently, to the recognition sequence and cleave the nucleic acid molecule wherein the nucleic acid molecule is labeled by a fluorescent label at a position such that upon the cleavage a cleaved fragment not bound by the transposase or integrase and carries the fluorescent label is released;
b) exposing the transposase or integrase protein and the nucleic acid molecule of step a) to a test agent;
c) measuring the fluorescence polarization of the label either during or following step b); and
d) comparing the fluorescence polarization measurement of step c) to that of at least one control to determine whether the test agent can modulate the nucleic acid cleavage activity of the transposase or integrase.

9. The method of claim 8, wherein the method is for identifying an agent that can modulate the nucleic acid cleavage activity of a transposase.

10. The method of claim 9, wherein the transposase is selected from the group consisting of Tn5 transposase, Tn10 transposase, and Mu transposase.

11. The method of claim 10, wherein the transposase is Tn5 transposase.

12. The method of claim 8, wherein the nucleic acid molecule is a DNA molecule between 2 and 60 nucleotides in length.

13. The method of claim 8, wherein the control is selected from the group consisting of (i) a positive cleavage control that comprises the transposase or integrase protein and the nucleic acid molecule under the conditions that allow the transposase or integrase to bind and cleave the nucleic acid molecule, (ii) a negative cleavage control that comprises the transposase or integrase protein and the nucleic acid molecule under the conditions that inhibit the nucleic acid cleavage activity of the transposase or integrase but allow the binding between the transposase or integrase and the nucleic acid molecule, and (iii) a pseudo-positive cleavage control that consists essentially of the fluorescently labeled nucleic acid molecule not bound by the transposase or integrase protein;
wherein the test agent is identified as a cleavage inhibitor if the fluorescence polarization measurement of step c) is comparable to that of the negative cleavage control, higher than that of the positive cleave control or that of the mock cleavage control, or both; and
wherein the test agent is identified as a cleavage enhancer if the fluorescence polarization measurement of step c) is lower than that of the positive cleave control.

14. The method of claim 13, wherein at least two controls are employed.

15. The method of claim 8, wherein the method is for identifying an agent that can inhibit the nucleic acid cleavage activity of a transposase or integrase.

16. The method of claim 8, wherein steps a) through d) are performed for at least 10 test agents simultaneously and the mean of the at least 10 fluorescence polarization measurements is employed as a control and wherein a substantially higher or lower fluorescence polarization measurement for a particular test agent than the control indicates that the agent is a cleavage modulator.

17. A method for identifying an agent that can modulate the binding between a transposase or integrase protein and its recognition sequence, the method comprising the steps of:
a) providing a transposase or integrase protein and a fluorescently labeled nucleic acid molecule that comprises a recognition sequence of the transposase or integrase under the conditions that allow the specific, non-covalent binding between the transposase or integrase protein and the recognition sequence but do not allow the transposase or integrase to cleave the nucleic acid molecule;
b) exposing the transposase or integrase protein and the nucleic acid molecule to a test agent;
c) measuring the fluorescence polarization of the label during or following step b); and
d) comparing the fluorescence polarization measurement of step c) to that of at least one control to determine whether the agent can modulate the binding between the transposase or integrase and its recognition sequence.

18. The method of claim 17, wherein the method is for identifying an agent that can modulate the binding between a transposase protein and its recognition sequence.

19. The method of claim 18, wherein the transposase is selected from the group consisting of Tn5 transposase, Tn10 transposase, and Mu transposase.

20. The method of claim 19, wherein the transposase is Tn5 transposase.

21. The method of claim 17, wherein the nucleic acid molecule is a DNA molecule between 2 and 60 nucleotides in length

22. The method of claim 17, wherein the control is selected from the group consisting of (i) a positive binding control that comprises the transposase or integrase protein and the nucleic acid molecule under the conditions that allow the transposase or integrase to bind to the nucleic acid molecule but do not allow the transposase or integrase to cleave the nucleic acid molecule and (ii) a negative binding control that consists essentially of the fluorescently labeled nucleic acid molecule not bound by the transposase or integrase protein;
wherein the test agent is identified as a binding inhibitor if the fluorescence polarization measurement of step c) is comparable to that of the negative binding control, lower than that of the positive binding control, or both; and
wherein the test agent is identified as a binding enhancer if the fluorescence polarization measurement of step c) is higher than that of the positive cleave control.

23. The method of claim 5, wherein both controls are employed.

24. The method of claim 17, wherein the method is for identifying an agent that can inhibit the binding between a transposase or integrase protein and its recognition sequence.

25. The method of claim 17, wherein steps a) through d) are performed for at least 10 test agents simultaneously and the mean of the at least 10 fluorescence polarization measurements is employed as a control and wherein a substantially higher or lower fluorescence polarization measurement for a particular test agent than the control indicates that the agent is a binding modulator.

26. A method for identifying an agent that can modulate the nucleic acid cleavage activity of a non-Tn5 transposase or integrase, the method comprising the steps of:
providing a Tn5 transposase DNA cleavage modulator as identified according to claim 8; and
testing whether the agent can modulate the non-Tn5 transposase or integrase activity.

27. A method for identifying an agent that can modulate the binding between a non-Tn5 transposase or integrase protein and its recognition sequence, the method comprising the steps of:
providing an agent that can modulate the binding between Tn5 transposase and its recognition sequence identified according to claim 17; and
testing whether the agent can modulate the binding between the non-Tn5 transposase or integrase and its corresponding recognition sequence.

28. A method for identifying an anti-HIV agent comprising the steps of:
providing an agent that can inhibit Tn5 transposase's DNA cleavage activity or binding between Tn5 transposase and its recognition sequence as identified according to claim 8 or 17; and
testing whether the agent can inhibit an activity of a HIV integrase, HIV integration, or both.

29. A method for inhibiting Tn5 transposase activity comprising the step of:
exposing Tn5 transposase to a compound defined by a formula selected from the group consisting of compounds 1-20 in FIG. 6.

30. A method for inhibiting HIV integrase's activity comprising the step of:
exposing HIV integrase to a compound defined by a formula selected from the group consisting of compounds 2, 4, 6, 10, 14, and 18 in FIG. 8 and compounds 10B-G in FIG. 9.

31. The method of claim 30, wherein the formula is selected from the group consisting of compounds 10 and 10B-G in FIG. 9.

32. A method for inhibiting HIV integration in cells comprising the step of:
exposing an HIV virus to a compound having the formula: 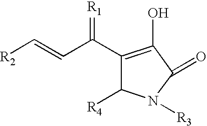
wherein R₁ is O or S; and R₂ to R₄ are identical or different and represent an aryl group or a substituted aryl group.

33. The method of claim 32, wherein R₁ is O.

34. The method of claim 32, wherein the aryl group or substituted aryl group is a phenyl group or substituted phenyl group.

35. The method of claim 34 wherein the substituted phenyl group is represented by 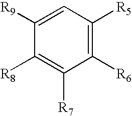
wherein R₅ to R₉ are identical or different and are selected from the group consisting of a hydrogen atom, a hydroxyl group, a halogen atom, a nitro group, an amino group, C≡N, S—H, and a carbon chain of 1-6 carbons, the carbon chain can be saturated, unsaturated, linear, or branched and can have heteroatoms attached as part of the chain or a side group wherein the heteroatoms are selected from the group consisting of F, Cl, Br, I, O, S, P, and N.

36. The method of claim 35, wherein R₂ is a phenyl group, R₃ is a phenyl group, a C₁₋₃ alkoxy group substituted phenyl group, or a trifluoro group substituted phenyl group, and R₄ is a nitro group substituted phenyl group.

37. The method of claim 32, wherein the compound is selected from the group consisting of compounds 10, 10B, and 10F in FIG. 9 and a compound having the formula [chemical structure]
